(19) United States
(12) Plant Patent Application Publication
(10) Pub. No.: US 2018/0084697 P1
(43) Pub. Date: Mar. 22, 2018

(54) Zamioculcas plant named 'Dowon'

(50) ;

(71) Applicant: Hyuk Jin Lee, Yongin (KR)

(72) Inventor: Hyuk Jin Lee, Yongin (KR)

(21) Appl. No.: 15/330,414

(22) Filed: Sep. 16, 2016

(51) Int. Cl. A01H 5/12

(52) U.S. Cl. PLT/373

(57) ABSTRACT

A new variety of Zamioculcas plant named ‘Dowon’ that is characterized by an erect vase-shaped habit, and stiffly-held compound, glossy leaves which emerge mid-green maturing to black, is described.

Genus and species: Zamioculcas zamiifolia.

Variety denomination: ‘Dowon’.

### CROSS-REFERENCE TO RELATED APPLICATIONS

A Community Plant Variety Rights application was filed with the Community Plant Variety Office of Feb. 16, 2015, File Number 2015/0379; and Plant Breeders Rights were granted for this variety with the Department of Plant Variety Protection—Korea Forest Service on Dec. 26, 2013, Registration Number 4721. This variety has not been made publicly available or sold more than one year prior to the filing date of the present application.

### BACKGROUND OF THE NEW PLANT

The present variety relates to a new and distinct variety of Zamioculcas commonly known as Aroid Palm or ZZ Plant. The new variety is known botanically as Zamioculcas zamiifolia and will be referred to hereinafter by the name ‘Dowon’.

Zamioculcas is a succulent plant with dark green glossy compound leaves which stores water in its thick petioles. Zamioculcas is native to tropical and sub-tropical east and southeast Africa and only one species, zamiifolia, is known. Zamioculcas is grown and used as a tropical or indoor foliage plant.

Zamioculcas plants grow from an underground tuber which also comprises the stem. All of the growth above ground consists of erect or semi-erect large pinnately compound leaves borne on stiff petioles. Each leaflet within the compound leaf is attached to a central rachis. The attachment is typically sessile or minutely petiolulate.

The new Zamioculcas variety named ‘Dowon’ was discovered in 2006 by the inventor at the inventor's nursery in Segok-dong, Seoul, South Korea. The inventor observed that a single plant of typically green-foliaged Zamioculcas had produced on one of its compound leaves a set of leaflets with uncharacteristically very dark green coloration, tending to darken further as the leaflets expanded. When fully expanded, the leaflets, rachis and petiole became entirely black or near-black. Zamioculcas may be propagated from individual leaflet cuttings. The inventor continued to observe the original plant for many months before carrying out the first asexual propagation in 2006 using black leaflets. The inventor was interested to know if the black leaflets would root and would produce new plants with the same characteristic of leaf blackening. The inventor observed that new foliage growth from the rooted black leaflets first emerged typically green in color but became black or nearly black as the compound leaf expanded and matured.

The inventor has repeated this process of reproduction from black leaves and has determined that the development of black plants as herein described is consistent. The inventor has determined that ‘Dowon’ reproduces true to type in successive generations of asexual reproduction via leaflet cuttings.

### SUMMARY

The following traits have been repeatedly observed and represent the distinguishing characteristics of the new Zamioculcas variety named ‘Dowon’. In combination these traits set ‘Dowon’ apart from its species and from all other varieties of Zamioculcas known to the inventor. ‘Dowon’ has not been tested under all possible conditions and phenotypic differences may be observed with variations in environmental, climatic and cultural conditions, however, without any variance in genotype:

1. Plants of ‘Dowon’ are erect and vase-shaped, consisting of stiffly held compound leaves.

2. The leaflets of the compound leaves of ‘Dowon’ are borne predominantly in opposite pairs. Some leaf pairs are sub-opposite or closely alternate.

3. The leaflets of ‘Dowon’ are very closely attached, either sessile or minutely petiolulate, to a central rachis.

4. The first emerging foliage growth of ‘Dowon’ is bright glossy mid-green in color.

5. As each new compound leaf ages over a period of 1 to 2 months, the color of the leaf darkens through dark olive green, then developing streaks or patches of very dark green tending to black, to eventually entirely black.

6. Mature compound leaves, including petioles, rachis and leaflets of ‘Dowon’ are entirely black.

7. The leaf surfaces of ‘Dowon’ are glossy.

8. The inflorescence of ‘Dowon’ emerges at or just above ground level and consists of a pale green spathe surrounding a cream-white spadix.

### DESCRIPTION OF THE PHOTOGRAPHS

The accompanying color photographs illustrate the overall appearance of ‘Dowon’showing the colors as true as it is reasonably possible to obtain in colored reproductions of this type. Colors in the photographs may differ from the color values cited in the detailed botanical description, which more accurately describe the actual colors of the new variety ‘Dowon’. Except for FIG. 9 which was taken in Miami, Fla., all photographs have been made from plants which have been grown by the inventor at the inventor's nursery in Seoul, South Korea. No growth regulators have been applied to any of the plants in the drawings.

FIG. 1 shows a mature plant of ‘Dowon’ in all stages of foliage color development.

FIG. 2 shows a plant of ‘Dowon’ which is fully mature and is not producing any new green foliage.

FIG. 3 depicts a close-up view of foliage of ‘Dowon’ as it begins to darken; the vein midribs are prominent at this stage.

FIG. 4 depicts a compound leaf of ‘Dowon’ which is in the mid-stage of turning from green to black.

FIG. 5 depicts a compound leaf of ‘Dowon’ which has matured to entirely black in color.

FIG. 6 depicts a leaflet of ‘Dowon’ in its semi-mature (olive green color) condition.

FIG. 7 depicts a leaflet of ‘Dowon’ in its mature (black color) condition.

FIG. 8 depicts a fresh inflorescence (spathe and spadix) of ‘Dowon’ and an unfertilized aged inflorescence with strongly recurved peduncle, spathe and spadix.

FIG. 9 depicts a six months old plant of ‘Dowon” which has been grown out of doors in a 1-gallon container in Miami, Fla. and from which the detailed botanical description has been made.

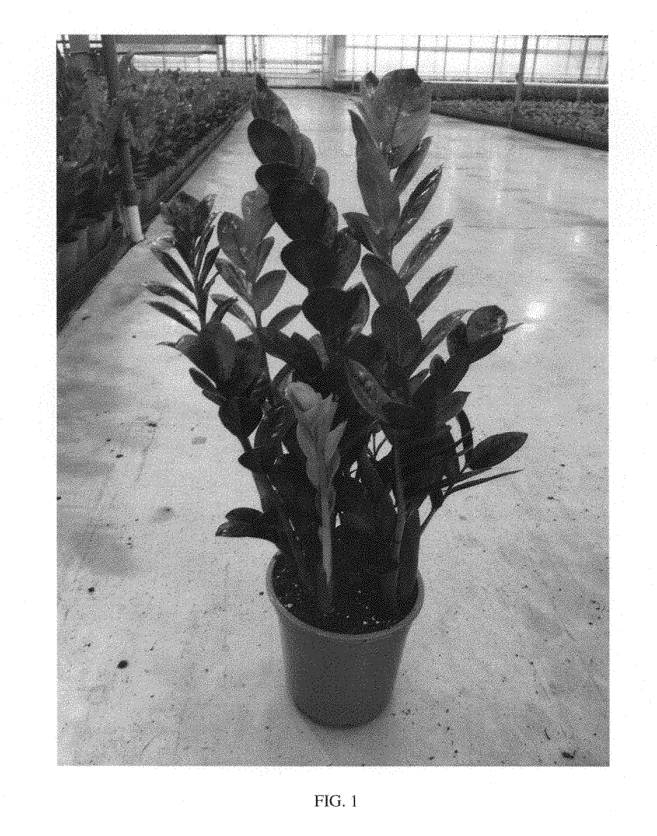

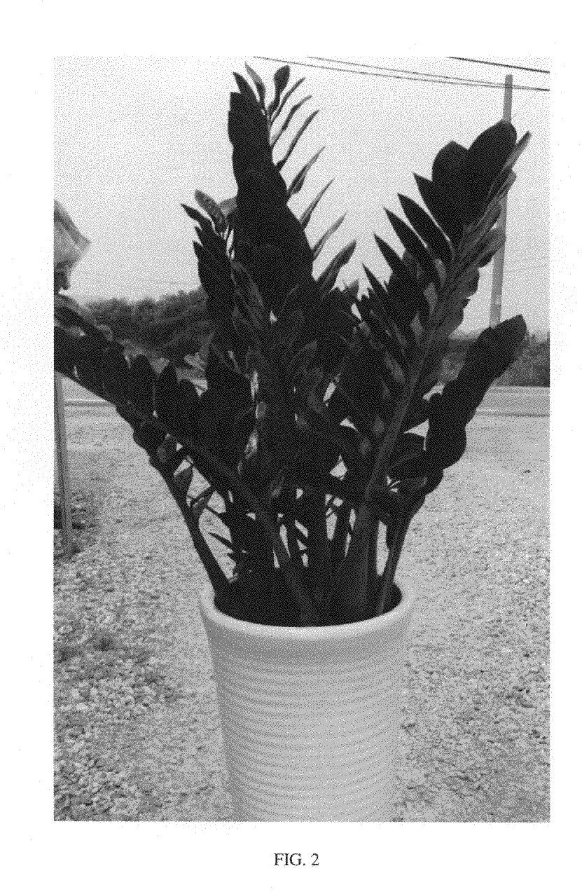

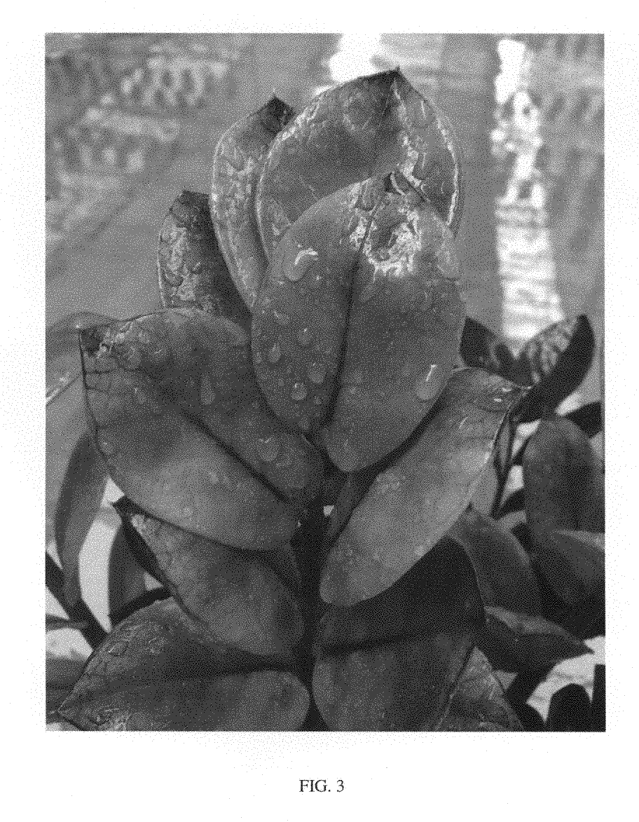

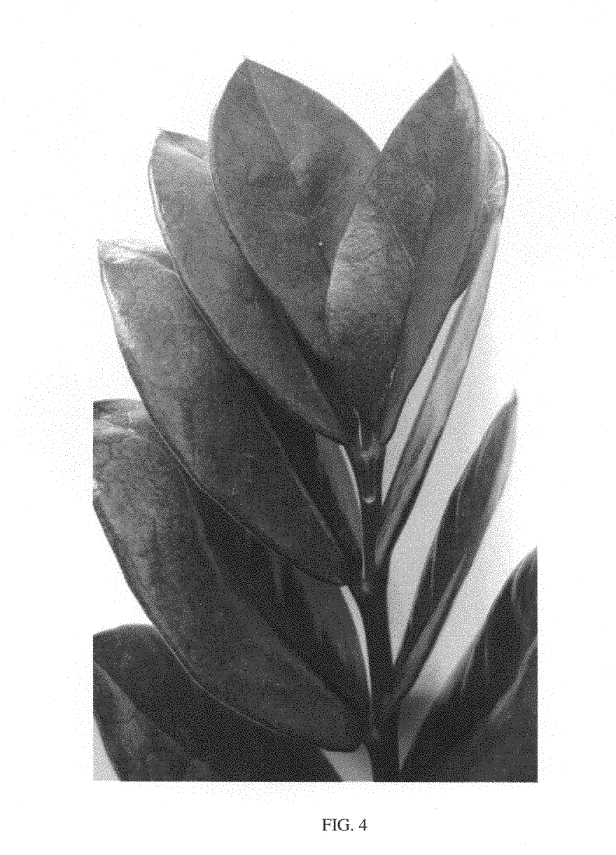

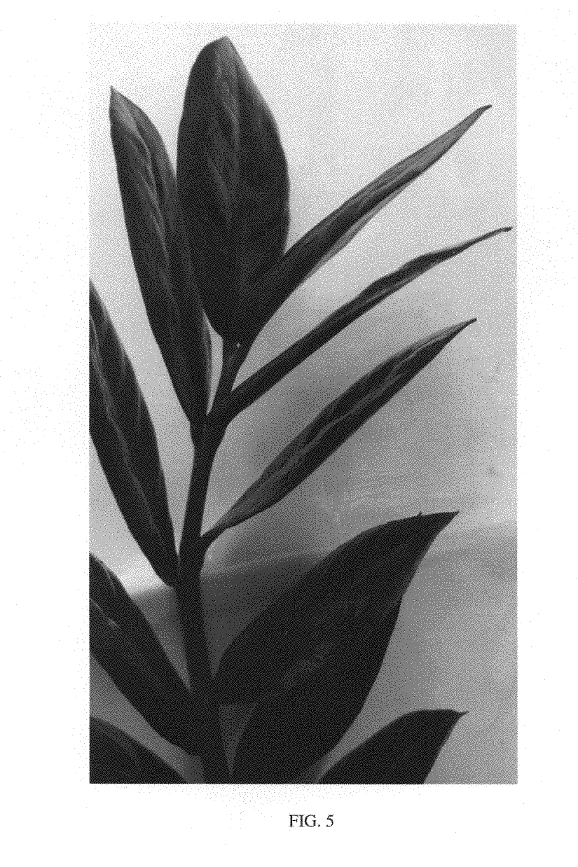

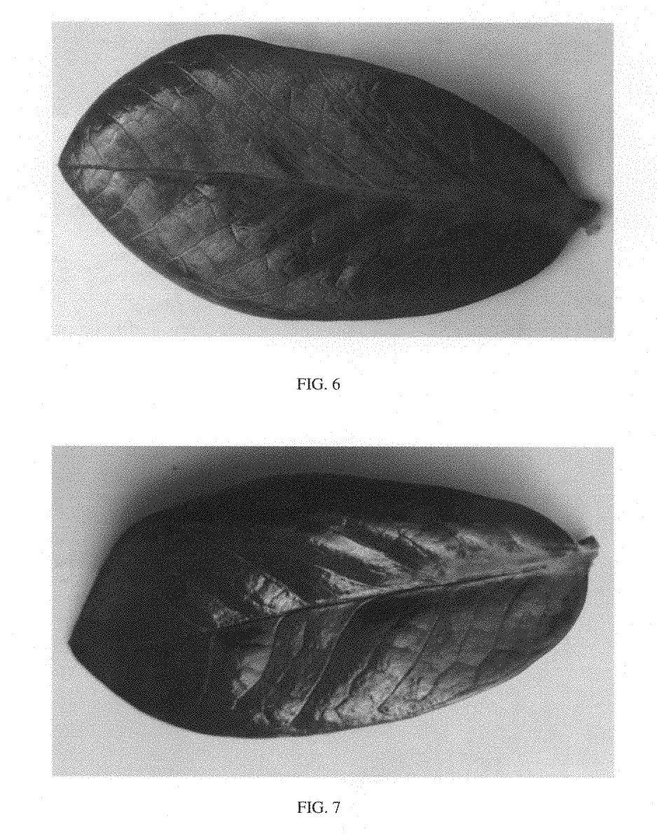

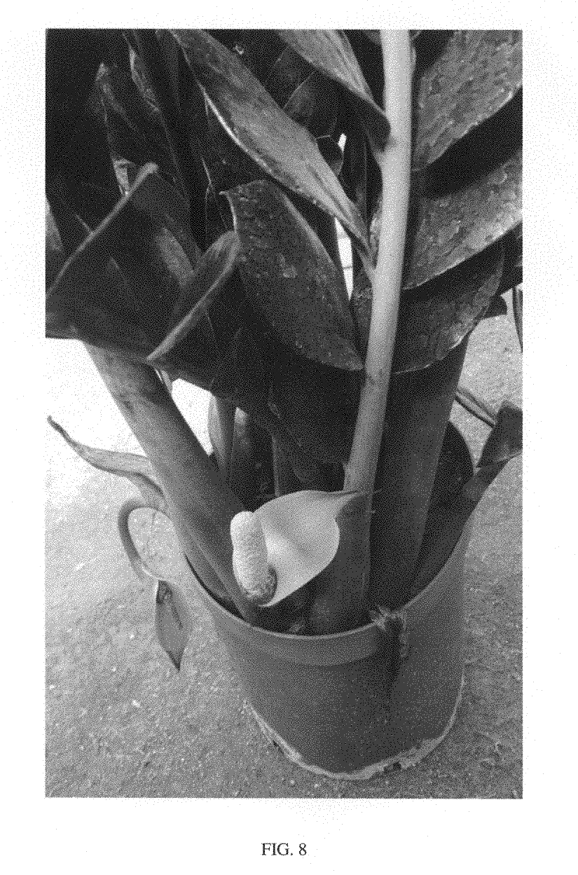

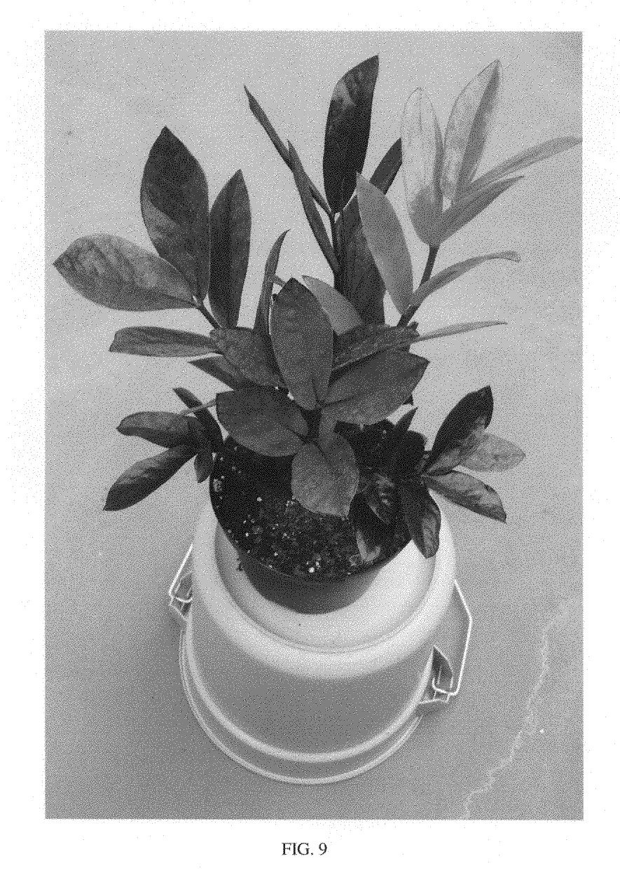

### DESCRIPTION OF THE NEW VARIETY

The following is a detailed description of the new Zamioculcas plant named ‘Dowon’. Data was collected in Santa Barbara, Calif. using six-month-old plants which were grown from a rooted leaflet from Miami, Fla. The description of the inflorescence of ‘Dowon’ was prepared from plants grown by the inventor in South Korea. The color determinations are in accordance with the 2007 edition of The Royal Horticultural Society Colour Chart, London, England, except where general color terms of ordinary dictionary significance are used. The growing requirements are similar to other Zamioculcas.
Botanical classification:
   Genus.—Zamioculcas.
   Species.—zamiifolia.
   Denomination.—‘Dowon’.
   Common name.—Aroid Palm.
Parentage: Sport from Zamioculcas zamiifolia (species, unpatented).
Plant:
   Plant height.—30.0 cm.
   Plant width.—22.0 cm in width.
   Roots description.—Fleshy; coarsely lateral branching; 1.0 mm to 3.0 mm in diameter; very fine root hairs; root color is 158A.
   Root development.—At soil temperatures 20° C. to 25° C., root initials are evident within 3 days; roots will reach the edge of a 1-gallon container in 21 days.
   Time to develop roots.—14 days at air and soil temperature of 25° C.
   Propagation type.—Asexual reproduction is accomplished by leaflet cuttings.
   Plant vigor.—Vigorous; prolific production of new leaves emerging from underground stem; 6 to 8 compound leaves per plant.
   Plant growth rate.—A 1-gallon container plant may be produced in 12 to 16 weeks from a 4.0-cm cell transplant, or in 5 to 6 months from insertion of a leaflet cutting.
   Plant growth habit and shape.—Upright and vase-shaped.
   Plant hardiness.—None; ‘Dowon’ does not tolerate freezing conditions.
   Cultural requirements.—‘Dowon’ thrives in high temperatures (above 70° F.), in full-sun or partial-shade.
   Use.—Interior decorative landscaping; outdoor landscaping in regions with no frost.
Tuber (underground stem):
   Shape and dimensions.—Short cylindrical; 5.0 cm in length, 2.0 cm to 3.0 cm in diameter.
   Surface texture.—Rough with root initials and leaf initials.
   Color.—Ranges between 158A and 165D.
Foliage (compound leaves):
   Quantity per plant.—6 to 8 compound leaves arise from underground stem.
   Petiole dimensions and shape.—Up to 10.0 cm in length; shape is cylindrical; diameter tapers from 10.0 mm at emergence from surface to 6.0 mm below lowest leaflet.
   Petiole surface and color.—Smooth, glabrous, strong and stiff; color is 152D (new green growth) and 202A (mature, black growth).
   Rachis dimensions.—Up to 15.0 cm in overall length with internode distance varying from 6.0 mm (between near-opposite leaflets) to 35.0 mm between leaflets furthest apart.
   Rachis surface and color.—Smooth, glabrous, strong and stiff; color is 152D (new green growth) and 202A (mature, black growth).
Foliage (leaflets):
   Attachment.—Sessile or minutely petiolulate.
   Dimensions (largest).—9.0 cm in length and 4.7 cm in width.
   Aspect.—Erect, held between 30° and 45° away from the rachis.
   Shape.—Obovate — elliptic.
   Apex.—Acuminate, point extends 1.5 mm.
   Base.—Cuneate — rounded, occasionally oblique.
   Surface.—Glossy (both surfaces).
   Color (both surfaces).—New green growth: 144A. Semi-mature growth: Mottled 139A against 146A. Mature black growth: 203B.
   Venation.—Pinnate, not prominent; appears as shallow depressions on adaxial surfaces and shallow ridges on abaxial surfaces).
   Vein color (new green growth, both surfaces).13 144A or very slightly lighter.
   Vein color (semi-mature growth, adaxial surface).—Midrib is 187A; elsewhere is 146A or slightly lighter.
   Vein color (semi-mature growth, abaxial surface).—Ranges between 139A and 146A.
   Vein color (mature growth, both surfaces).—203B. Petiolules (where present): Dimensions: Length is 1.0 mm to 3.0 mm and diameter is 1.0 mm to 3.0 mm. Shape: Sulcate. Surface texture: Smooth. Color: 144A (new green growth); 139A (semi-mature growth); and 203B (mature black growth).
Inflorescence:
   General.—Spadix flowers not closely observed; pollination has not been observed.
   Spathe.—Appearance: Short cylindrical tube with longer ovate blades, 5.0 cm to 6.0 cm in length and 4.0 cm to 5.0 cm in width. Length: 1.5 cm. Diameter: 1.5 cm. Apex: Acuminate, point extends up to 1.0 cm. Base: Sheathing. Spathe color (both surfaces): Ranges between NN155C and 145C. With age: Contracts with age.
   Spadix.—Shape: Tapering cylindrical. Length: 6.0 cm. Diameter: 1.5 cm at widest, narrowed to 1.0 cm at the base. Color: NN155D. With age: Contracts with age.
   Peduncle.—Appearance: Short and erect. Length: Approximately 2.0 cm. Diameter: 0.5 cm. Color: N189B. With age: Peduncle extends and becomes strongly recurved, approximately 8.0 cm in length, 0.5 cm in diameter, and color fades to N187B.
Resistance or susceptibility to pests and diseases: ‘Dowon’ has been observed to be less attractive to common pests which affect indoor plants including mites (Tetranychidae), aphids (Aphididae) and mealybugs (Pseudococcidae).

### COMPARISON TO PARENTAL LINE

‘Dowon’ may be compared with its sport parent, the species Zamioculcas zamiifolia (species, unpatented). Whereas the compound leaves (including leaflets, rachis and petiole) of Zamioculcas zamiifolia are green in color, a mature plant of ‘Dowon’ is uniformly black or near-black. In addition, the inventor has observed that ‘Dowon’ is less attractive to common pests which affect indoor plants including mites, aphids and mealybugs.

‘Dowon’ may also be compared with the species Zamioculcas zamiifolia by the rate of growth of new leaves arising from a rooted leaf cutting. Altogether, the inventor has observed that a mature (60.0 cm in height) fully-black plant of ‘Dowon’ may take 7 to 8 months to produce compared with 3 to 4 months for production of green Zamioculcas zamiifolia.

No commercial varieties of Zamioculcas which exhibit black foliage are known to the inventor.

I claim:

1. A new and distinct variety of Zamioculcas plant named ‘Dowon’ as illustrated and described herein.
